(19) United States
(12) Plant Patent Application Publication
(10) Pub. No.: US 2002/0129410 P1
(43) Pub. Date: Sep. 12, 2002

(54) Strawberry plant named La Conchita

(76) Inventors: Amado Q. Amorao, Camarillo, CA (US); Arnoldo Solis JR., Oxnard, CA (US); Michael Ferguson, Chatsworth, CA (US)

Correspondence Address: PENNIE & EDMONDS LLP, 1155 Avenue of the Americas, New York, NY, 10036-2711, US

(21) Appl. No.: 09/975,824

(22) Filed: Oct. 12, 2001

(51) Int. Cl. A01H 5/00

(52) U.S. Cl. PLT/208

(57) ABSTRACT

This invention relates to a new and distinct variety of strawberry named La Conchita. The variety is similar to the varieties Coronado and San Miguel. The variety is distinguished from Coronado and San Miguel, in particular, the plant is medium dense, shape of terminal leaflet is obtuse, petiole pubescence is dense, predominant fruit shape is cordate, insertion of achenes is level with surface, and fruit acidity is weak.

### 1. BACKGROUND OF THE INVENTION

The new variety originated as a result of a controlled cross between the strawberry plants Ana Maria (U.S. Plant Pat. No. 11,035) and Balboa (U.S. Plant Pat. No. 9,130) in an ongoing breeding program, and was discovered as a seedling in a controlled breeding plot in Ventura County, California in February, 1996. The original seedling of the new cultivar was asexually propagated by stolons in McArthur, Shasta County, California. Propagules were transplanted to a controlled breeding plot in Ventura County, California, where the variety was identified and selected for further evaluation. La Conchita was subsequently asexually propagated and underwent further testing in Ventura County, California for six years. This propagation and testing has demonstrated that the combination of traits disclosed herein which characterize the new variety are fixed and retained true to type through successive generations of asexual reproduction.

### 2. SUMMARY OF THE INVENTION

The present invention relates to a new and distinct variety of strawberry named La Conchita. The variety is botanically identified as Fragaria x ananassa. The new variety is distinguished from other varieties by a number of characteristics as set forth in Tables 1-4.

### 3. COMPARISON TO SIMILAR VARIETIES

The varieties which we believe to be similar to La Conchita from those known to us are Coronado (U.S. Plant Pat. No. 10,221) and San Miguel (U.S. Plant Pat. No. 10,642). There are several characteristics of the new variety that are different from, or not possessed by Coronado and San Miguel. The new variety is medium dense, shape of terminal leaflet is obtuse, petiole pubescence is dense, predominant fruit shape is cordate, insertion of achenes is level with surface, and fruit acidity is weak

### 4. BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows the leaves of the plant.

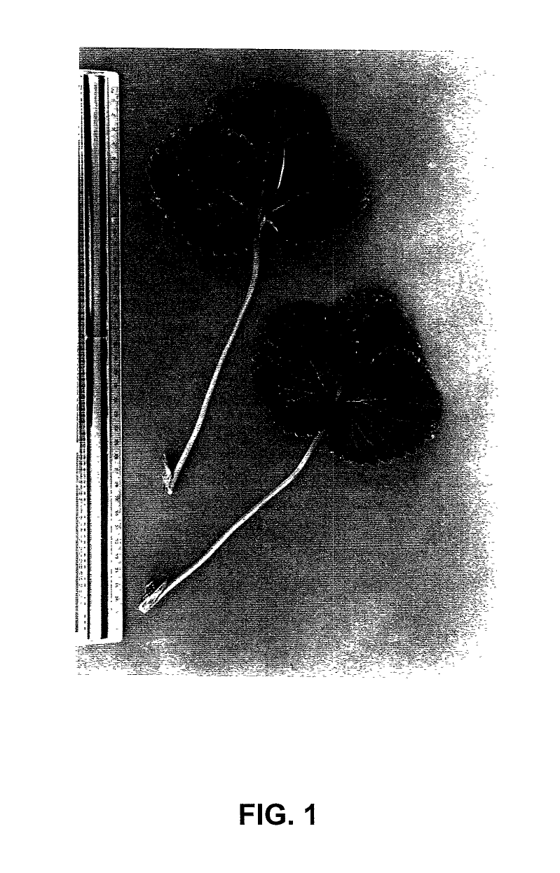

FIG. 2 shows the upper side and the under side of the flowers.

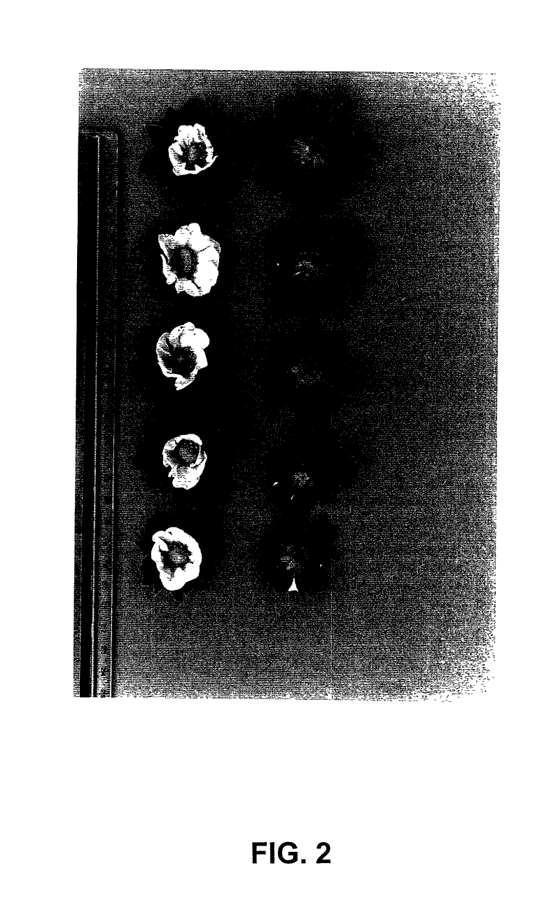

FIG. 3 shows a close-up of the fruit.

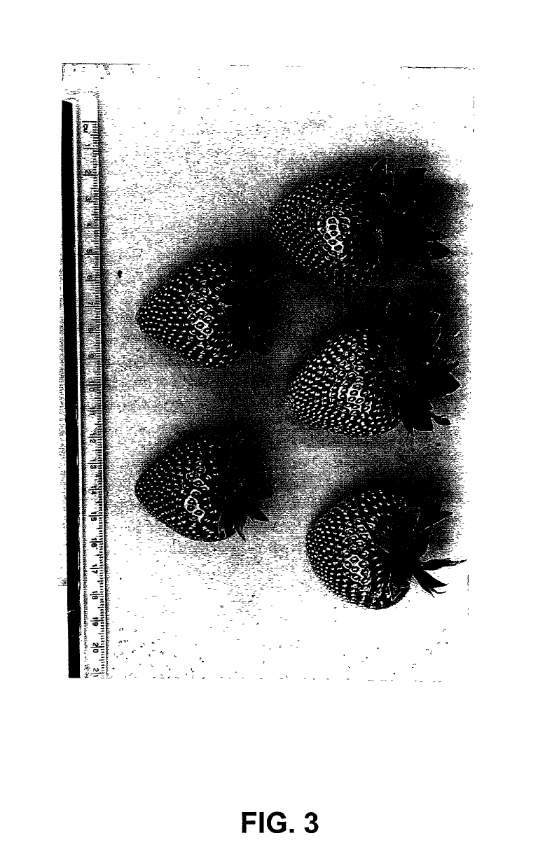

FIG. 4 shows the fruit in longitudinal cross-section.

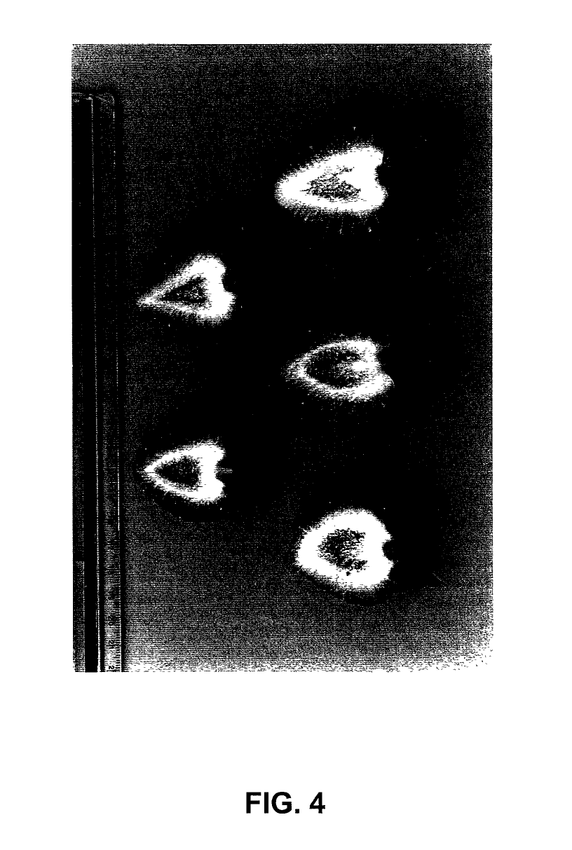

### 5. DESCRIPTION OF THE NEW VARIETY

The following detailed description of the new variety is based upon observations taken of plants and fruit grown in Ventura County, California, U.S.A. This description is in accordance with UPOV terminology. Observations of La Conchita, Coronado and San Miguel were taken in side by side comparison in the year 2001. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### 5.1 PROPAGATION

The new variety is principally propagated by way of stolons. Although propagation by stolons is presently preferred, other known methods of propagating strawberry plants may be employed.

### 5.2 CHARACTERISTICS OF THE NEW VARIETY

Information on the new variety is presented in Tables 1, 2, 3 and 4. In the tables, the flowers described are secondary flowers except where indicated. The petal color of La Conchita is white (155C in the RHS Colour Chart). The fruit described is the secondary fruit on one-year-old plants. Fruit and flower measurements are an average of both primary and secondary fruit and flowers.

Table 1 provides information on the plant and fruit characteristics of the new variety La Conchita compared with characteristics of Coronado and San Miguel. Table 2 provides additional information of the plant and fruit characteristics of the new variety La Conchita compared with characteristics of the varieties Coronado and San Miguel. Table 3 provides reactions of the new variety to stresses, pests and diseases compared with reactions of the varieties Coronado and San Miguel. Table 4 provides isozyme characteristics of the new variety as compared to that of the varieties Coronado and San Miguel.

TABLE 1
DETAILED COMPARISON OF LA CONCHITA, CORONADO AND
SAN MIGUEL

| | La Conchita | Coronado | San Miguel |
|---|---|---|---|
| Plant Characteristics | | | |
| Height of Plant (cm) | 19.5 | 20.7 | 22.8 |
| Spread of Plant (cm) | 26.0 | 28.7 | 29.4 |
| Number of Crowns | 3.4 | 4.6 | 4.4 |
| Leaf Characteristics | | | |
| Terminal Leaflet Length (cm) | 5.6 | 6.1 | 6.2 |
| Terminal Leaflet Width (cm) | 6.2 | 6.3 | 5.8 |
| Terminal Leaflet Length/Width | 0.91 | 0.97 | 1.07 |
| # Teeth/Terminal Leaflet | 22.7 | 21.6 | 20.7 |
| Color of upper side | dark green | light to | medium to |
| | 147A | medium | dark green |
| | | green | 147A |
| | | 137A | |
| Color of under side | light to | light to | light to |
| | medium | medium | medium |
| | green | green | green |
| | 139C | 137B | 138B |
| Petiole Length (cm) | 14.9 | 16.3 | 16.0 |
| Petiole Color | 149A | 149A | 149A |
| Bract Frequency | 0% | 0% | 25% |
| | | | typically |
| | | | paired |
| Stipule Length (cm) | 2.9 | 3.4 | 2.9 |
| Stipule Width (cm) | 1.0 | 1.2 | 1.1 |
| Flower Characteristics | | | |
| Petal Length (cm) | 1.7 | 1.6 | 1.4 |
| Petal Width (cm) | 1.6 | 1.7 | 1.5 |
| Petal Length/Width Ratio | 1.06 | 0.93 | 0.98 |
| Flower Diameter (cm) | 3.2 | 3.4 | 2.7 |
| Calyx Diameter (cm) | 4.4 | 4.4 | 4.1 |
| Fruiting Truss | | | |
| Length (cm) | 21.8 | 22.1 | 25.7 |
| Fruit Characteristics | | | |
| Fruit Length (cm) | 4.2 | 4.3 | 4.3 |
| Fruit Width (cm) | 4.1 | 3.9 | 4.1 |
| Fruit Length/Width Ratio | 1.04 | 1.11 | 1.07 |
| Average Berry Weight (g) | 27.0 | 23.6 | 23.2 |
| External Color | 45A | 46A | 53A |
| Internal Color | orange red | red | dark red |
| | and white | 41A | 45A |
| | (34A and | | |
| | 155 C) | | |
| Average % brix | 8.1 | 7.1 | 7.1 |
| Brix/Acid Ratio | 10.75 | 9.11 | 9.33 |
| Achene Coloration | 60A to 15B | 53A to | 53A to 14B |
| | | 151B | |
| Yield (g/plant) | 695 | 598 | 557 |

TABLE 2
CHARACTERISTICS OF LA CONCHITA, CORONADO AND SAN
MIGUEL

| | La Conchita | Coronado | San Miguel |
|---|---|---|---|
| Plant | | | |
| Habit | globose | globose | flat globose |
| Density | dense | open | open |
| Vigor | medium | medium | medium |
| Leaf | | | |
| Shape in cross | slightly concave | slightly concave | concave |
| section | | | |
| Interveinal | strong | strong | strong |
| blistering | | | |
| Glossiness | strong | strong | strong |
| Number of | more than three | more than three | three only |
| leaflets | on at least 5 out | on up to 5 out of | |
| | of 10 leaves | 10 leaves | |
| Terminal leaflet | flat | flat to cupped | flat to revolute |
| margin profile | | | |
| Terminal leaflet | rounded | obtuse | rounded |
| shape of base | | | |
| Terminal leaflet | obtuse | rounded | rounded |
| shape of teeth | | | |
| Stipule | sparse to | sparse | sparse |
| pubescence | medium | | |
| Petiole | dense | medium | sparse to |
| pubescence | | | medium |
| Petiole pose of | outward to | outward | outward |
| hairs | downward | | |
| Stolon | | | |
| Amount | many | medium to many | many |
| Anthocyanin | medium | weak to medium | weak to |
| coloration | | | medium |
| Thickness | medium | medium | thin to medium |
| Pubescence | medium to dense | sparse to medium | sparse to |
| | | | medium |
| Inflorescence | | | |
| Position relative | level to above | level to above | above |
| to foliage | | | |
| Diameter of calyx | larger | larger | larger |
| relative to corolla | | | |
| on secondary | | | |
| flowers | | | |
| Diameter of inner | smaller | same size to | same size |
| calyx relative to | | larger | |
| outer on | | | |
| secondary flowers | | | |
| Spacing of petals | touching to | touching to | overlapping |
| | overlapping | overlapping | |
| Fruiting Truss | | | |
| Attitude at first | prostrate | semi-erect to | prostrate |
| picking | | prostrate | |
| Fruit | | | |
| Predominant | cordate | conical to wedged | conical to |
| shape | | | cylindrical |
| Difference in | slight | moderate | moderate |
| shapes between | | | |
| primary and | | | |
| secondary fruits | | | |
| Band without | very narrow | narrow | very narrow |
| achenes | | | |
| Unevenness of | weak | weak to medium | weak to |
| surface | | | medium |
| Evenness of color | slightly uneven | even | even |
| uneven | to even | | |
| Glossiness | medium to | medium to strong | medium |
| | strong | | |
| Insertion of | level with the | below surface | below surface |
| achenes | surface | | |
| Insertion of calyx | level to set above | `level to set above | set above |
| Pose of the calyx | reflexed | spreading | spreading to |
| segments | | | reflexed |
| Size of calyx in | same size to | larger | same size |
| relation to fruit on | larger | | |
| secondary fruit | | | |
| Adherence of | strong | medium | strong |
| calyx | | | |
| Firmness of flesh | medium | firm | firm |
| Evenness of flesh | uneven | slightly uneven | even |
| color | | | |
| Distribution of | only marginal | marginal and | marginal and |
| flesh color | | central | central |
| Hollow center | small to medium | medium | medium |
| size | | | |
| Sweetness | strong | weak | strong |
| Texture when | fine | fine | fine |
| tasted | | | |
| Acidity | weak | strong | medium |
| Time of | early-January | early-January | early-January |
| Flowering | | | |
| Harvest Interval | early-February to | early-February to | early-February |
| in 2001 | mid-June | mid-June | to mid-June |
| Type of Bearing | partially | partially | partially |
| | everbearing | everbearing | everbearing |

TABLE 3
5.3 REACTION TO STRESS, PESTS AND DISEASE FOR
LA CONCHITA, CORONADO AND SAN MIGUEL

| | La Conchita | Coronado | San Miguel |
|---|---|---|---|
| Reaction to Stress | | | |
| high pH | moderately | moderately | moderately |
| | resistant | resistant | resistant |
| high soil salt levels | moderately | moderately | moderately |
| | resistant | resistant | resistant |
| Reaction to Pests | | | |
| Tetranychus urticae | moderately | moderately | moderately |
| | resistant | resistant | resistant |
| Lygus hesperus | susceptible | susceptible | susceptible |
| Reaction To Diseases | | | |
| Botrytis fruit rot | moderately | moderately | moderately |
| | susceptible | susceptible | susceptible |
| Powdery mildew | moderately | moderately | moderately |
| | resistant | resistant | resistant |
| Verticillium wilt | susceptible | susceptible | susceptible |
| Strawberry Mottle Virus | moderately | moderately | moderately |
| | resistant | resistant | resistant |
| Xanthomonas fragariae | moderately | moderately | moderately |
| | susceptible | susceptible | susceptible |

### 5.4 ISOZYME ANALYSIS

In addition to the morphological description above, the new cultivar La Conchita has been analyzed to obtain an indication of its genetic makeup to provide further means for identifying the new variety and distinguishing it from other somewhat similar and/or related strawberry varieties. Specifically, leaf samples of La Conchita, Coronado, and San Miguel were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR LA CONCHITA, CORONADO AND
SAN MIGUEL

| Locus | La Conchita | Coronado | San Miguel |
|---|---|---|---|
| PGI | A4 | A2 | A4 |
| LAP | B3 | B3 | B3 |
| PGM | C2 | C4 | C2 |

What is claimed is:

1. A new and distinct variety of strawberry plant, substantially as shown and described.
